(19) United States
(12) Patent Application Publication
(10) Pub. No.: US 2017/0238979 A1
(43) Pub. Date: Aug. 24, 2017

(54) BONE FIXATION SYSTEM AND A PLATE THEREFOR

(71) Applicant: Austofix Group Limited, North Plympton (AU)

(72) Inventor: Ewen Laird, North Plympton (AU)

(73) Assignee: Austofix Group Limited, North Plympton (AU)

(21) Appl. No.: 15/521,596

(22) PCT Filed: Oct. 23, 2015
(86) PCT No.: PCT/AU2015/000632

§ 371 Date: Apr. 24, 2017

(30) Foreign Application Priority Data
Oct. 24, 2014 (AU) 2014904269

(51) Int. Cl. A61B 17/80; A61B 17/86

(52) U.S. Cl. CPC A61B 17/8052; A61B 17/8605; A61B 2017/00526

(57) ABSTRACT

The present disclosure relates to a bone fixation system for orthopaedic surgery. In one form, the system comprises a screw and a plate, the plate comprising at least one through opening, the through opening comprising a screw inlet, and a through bore which tapers inwardly as it extends through the plate from the screw inlet, the plate further comprising a plurality of splines which further define the through opening by projecting into the through bore, each spline extending longitudinally through the through opening, and comprising a top land which extends from the screw inlet and along the spline, and wherein in use, at least a portion of a head of the screw will engage with the top lands of the splines. A plate for a bone fixation system is also disclosed.

### PRIORITY DOCUMENTS

The present application claims priority from Australian Provisional Patent Application No. 2014904269 titled “A BONE FIXATION SYSTEM AND A PLATE THEREFOR” and filed on 24 Oct. 2014, the content of which is hereby incorporated by reference in its entirety.

### INCORPORATION BY REFERENCE

The following co-pending patent application is referred to in the following description: PCT/AU2013/000536 titled “BONE FIXATION DEVICE”, which claims priority from Australian Provisional Patent Application No. 2012902106.

The content of this application is hereby incorporated by reference in its entirety.

### TECHNICAL FIELD

This disclosure relates to the field of medical devices, in particular, in the field of orthopaedic surgery using bone fixation systems comprising bone fixation elements such as pegs and screws, and a bone fixation plate.

In a particular form the present disclosure relates to the bone fixation plate.

### BACKGROUND

Internal fracture fixation is often necessary when a fracture occurs within the human body. A goal of the fixation is to provide immediate stabilization to the fracture once it has been reduced to its correct alignment. Another goal is to encourage rapid healing and return mobility to an individual thus preventing muscular atrophy. Metallic plates (CoCrMo alloy, stainless steel or titanium and it's alloys) can be fixed to the outside of the bone using a variety of fixation elements such as screws, pins, nails, pegs and wires, selectively placed around a fracture site.

Many current bone fixation systems use locking screws to fix the plates to the bone. A locking screw is so named because it not only screws into the bone, but also locks into the plate independently. Locking screws prevent toggling of the screw due to lateral forces and minimise any compression of the plate onto the bone. Some plates use fixed angle locking screws, which are fixed in a particular orientation relative to the plate, whilst others use variable angle locking screws that can be fixed to the bone within a range of angles, relative to the plate. There are various ways of allowing a screw to be inserted at variable angles, but many conventional variable angle screws have a tapered ‘locking’ thread, which engages with an internal thread of a hole in the plate. The variable angle screw can be inserted at a surgeon-directed angle through the plate, which will usually result in the screw cross-threading in the screw hole, and deforming the internal thread of the screw hole. The range of permissible angles can vary, but may be, for example, ±15 degrees. Other forms of variable angle locking include the use of collets, friction, or holes with intermittent threaded sections

Fixed angle locking screws are generally considered to provide increased stability, relative to variable angle screws. Variable angle screws can suffer from lack of stability from some directions, and typically also have a relatively low back-out torque, such that if the screw is loosened by a fraction of a turn, the grip of the male thread is lost and the screw can begin to toggle in the hole.

On the other hand, the advantage of a variable angle screw is that it allows the surgeon to better tailor the application of the plate system to the specific nature of the bone fracture.

It is against this background and the problems and difficulties associated therewith that the present invention has been developed.

Certain objects and advantages of the present invention will become apparent from the following description, taken in connection with the accompanying drawings, wherein, by way of illustration and example, an embodiment of the present invention is disclosed.

### SUMMARY

Where terms such as ‘spline’, ‘top land’, ‘bottom land’ and ‘fillet’ are used throughout this specification, the meaning of these terms is to be construed in accordance with gear and spline nomenclature.

According to a first aspect, there is provided a bone fixation system comprising a screw and a plate, the plate comprising at least one through opening, the through opening comprising a screw inlet, and a through bore which tapers inwardly as it extends through the plate from the screw inlet, the plate further comprising a plurality of splines which further define the through opening by projecting into the through bore, each spline extending longitudinally through the through opening, and comprising a top land which extends from the screw inlet and along the spline, and wherein in use, at least a portion of a head of the screw will engage with the top lands of the splines.

According to a further aspect, there is provided a plate for a bone fixation system comprising the plate and at least one screw, the plate comprising at least one through opening, the through opening comprising a screw inlet, and a through bore which tapers inwardly as it extends through the plate from the screw inlet, the plate further comprising a plurality of splines which further define the through opening by projecting into the through bore, each spline extending longitudinally through the through opening, and comprising a top land which extends from the screw inlet and along the spline.

In one form, the splines are equi-spaced.

In one form, the splines are substantially identical.

In one form, each spline comprises a symmetrical cross-sectional shape. In one form, in an alternative, each spline comprises an asymmetrical cross-sectional shape.

In one form, each spline extends through the through bore by the most direct route. That is to say, substantially parallel to a central axis of the through bore.

In one form, in an alternative, each spline does not extend through the through bore by the most direct route, but extends around a wall of the through bore as it extends through the through bore. In other words, each spline spirals about the through bore.

In one form, each top land comprises a width which narrows as the spline extends through the plate from the screw inlet.

In one form, in an alternative, a width of each top land remains substantially constant as the spline extends through the plate from the screw inlet.

In one form, the plate comprises five (5) equi-spaced splines, which further define the through opening by projecting into the through bore.

In one form, the top land of each spline tapers inwardly as it extends through the plate from the screw inlet.

In one form, each spline deepens as it extends longitudinally through the through opening. In one form, in an alternative, the top land of each spline extends parallel to the through bore.

In one form, an edge of the plate surrounding the screw inlet comprises a lead-in portion which tapers both inwardly as it extends from the screw inlet to the splines, and with a higher degree of taper than the through bore.

In one form, in an alternative, an edge of the plate surrounding the screw inlet comprises a filleted (or rounded) lead-in portion.

In one form, each spline comprises a flat side to either side of the top land.

In one from, the spline sides are of different lengths and/or degrees of inclination.

In one form, in an alternative, each spline comprises a fillet to either side of the top land, where each fillet blends into a bottom land which separates adjacent splines.

In one form, the fillet is concave and shaped like an arc of a circle. A radius of this fillet arc will hereinafter be referred to as the ‘fillet arc radius’.

In one form, the bottom land is concave and shaped like an arc of a circle. A radius of this bottom land arc will hereinafter be referred to as the ‘bottom land arc radius’.

In one form, the fillet arc radius and the bottom land arc radius are different.

In one form, the bottom land arc radius is greater than the fillet arc radius.

In one form, the screw is a poly-axial locking screw.

In one form, the screw comprises a head and a shank, wherein at least a portion of the head comprises a locking thread for engagement with the top lands of the splines.

In one form, the screw is comprised of a harder material that the plate, so that in use the locking thread cuts a thread in the plate.

In one form, at least a portion of the head comprises a multi start locking thread.

In one form, at least a portion of the head comprises a triple start locking thread.

In one form, the screw is substantially as disclosed in PCT/AU2013/000536, titled “BONE FIXATION DEVICE”. This screw comprises a triple start thread with a short parallel section. This provides for improved stability, particularly when the angle of screw inclination relative to the plate is high.

In one form, in an alternative, the screw is a poly-axial non-locking screw.

In one form, the poly-axial non-locking screw is comprised of a harder material than the plate.

In one form, the plate is manufactured by a 3D printing process.

In one form, the plate is not flat, but comprises at least one curve. In one form, the plate comprises a plurality of curves. In one faun, the plate comprises convex and/or concave curves.

In one form, the or each through hole does not extend perpendicular to a plate surface.

According to a further aspect, there is provided a bone fixation system comprising a screw and a plate, where the screw is comprised of a harder material than the plate, and wherein the plate comprises at least one through opening, the through opening comprising a screw inlet, and a through bore which tapers inwardly as it extends through the plate from the screw inlet, the plate further comprising five equi-spaced splines which further define the through opening by projecting into the through bore, each spline extending longitudinally through the through opening, and comprising a top land which extends from the screw inlet and along the spline, and wherein in use, at least a portion of a head of the screw comprises a triple start thread for cutting into the top lands of the splines.

According to a further aspect, there is provided a method for forming a plate of the above described type, the method comprising the steps of creating the through bore in the plate which tapers inwardly as it extends through the plate from the screw inlet, and then relieving a wall of the through bore identically at a plurality of equi-spaced locations so as to form the splines (i.e. the splines are remnants of the original through bore).

A detailed description of one or more embodiments of the invention is provided below along with accompanying figures that illustrate by way of example the principles of the invention. While the invention is described in connection with such embodiments, it should be understood that the invention is not limited to any embodiment. On the contrary, the scope of the invention is limited only by the appended claims and the invention encompasses numerous alternatives, modifications and equivalents. For the purpose of example, numerous specific details are set forth in the following description in order to provide a thorough understanding of the present invention.

For ease of description, a bone fixation system embodying the present invention is described below in its usual assembled position as shown in the accompanying drawings, and terms such as front, rear, upper, lower, horizontal, longitudinal etc., may be used with reference to this usual position. However, the bone fixation system may be manufactured, transported, sold, or used in orientations other than that described and shown here.

The present invention may be practiced according to the claims without some or all of these specific details. For the purpose of clarity, technical material that is known in the technical fields related to the invention has not been described in detail so that the present invention is not unnecessarily obscured.

### BRIEF DESCRIPTION OF DRAWINGS

Embodiments of the present invention will be discussed with reference to the accompanying drawings wherein:

FIG. 1 is an isometric view of a bone fixation system comprising a plurality of variable angle screws driven through a bone fixation plate;

FIG. 2 is a plan view of the bone fixation plate of FIG. 1;

FIG. 3 is an isometric detail view of a through opening in the bone fixation plate of FIG. 2;

FIG. 4 is a plan view of the through opening of FIG. 3;

FIG. 5 is a cross-sectional view taken at A-A in FIG. 4;

FIG. 6 is a side view of a screw from the bone fixation system of FIG. 1;

FIG. 7 is an isometric view of the screw of FIG. 6 passing through a portion of the bone fixation plate with no deviation (i.e. so that a longitudinal axis of the screw is aligned with a central axis of the through opening);

FIG. 8 is a cross-sectional view through the bone fixation plate of FIG. 7, further illustrating the screw passing there through;

FIG. 9 is an isometric view of the screw of FIG. 6 passing through a portion of the bone fixation plate with 5 degrees of deviation (i.e. so that a longitudinal axis of the screw is tilted 5 degrees with respect to a central axis of the through opening);

FIG. 10 is a cross-sectional view through the bone fixation plate of FIG. 9, further illustrating the screw passing there through;

FIG. 11 is an isometric view of the screw of FIG. 6 passing through a portion of the bone fixation plate with 10 degrees of deviation;

FIG. 12 is a cross-sectional view through the bone fixation plate of FIG. 11, further illustrating the screw passing there through;

FIG. 13 is an isometric view of the screw of FIG. 6 passing through a portion of the bone fixation plate with 15 degrees of deviation;

FIG. 14 is a cross-sectional view through the bone fixation plate of FIG. 13, further illustrating the screw passing there through;

FIG. 15 is an isometric view of the screw of FIG. 6 passing through a portion of the bone fixation plate with 20 degrees of deviation;

FIG. 16 is a cross-sectional view through the bone fixation plate of FIG. 15, further illustrating the screw passing there through;

FIG. 17 is an isometric view of a variable angle (poly-axial) non-locking screw passing through a portion of the bone fixation plate with no deviation (i.e. so that a longitudinal axis of the screw is aligned with a central axis of the through opening);

FIG. 18 is a cross-sectional view through the bone fixation plate of FIG. 17, further illustrating the screw passing there through;

FIG. 19 is a cross-sectional view through the bone fixation plate, further illustrating a variable angle non-locking screw passing there through with 20 degrees of deviation (i.e. so that a longitudinal axis of the screw is tilted 20 degrees with respect to a central axis of the through opening);

FIG. 20 an isometric detail view of a through opening according to a further embodiment;

FIG. 21 is a plan view of the through opening of FIG. 20;

FIG. 22 is an isometric detail view of a through opening according to a further embodiment;

FIG. 23 is a plan view of the through opening of FIG. 22;

FIG. 24 is an isometric view of a bone fixation plate according to a further embodiment; and

FIG. 25 is a cross-sectional view taken lengthwise through the bone fixation plate of FIG. 24.

In the following description, like reference characters designate like or corresponding parts throughout the figures.

### DESCRIPTION OF EMBODIMENTS

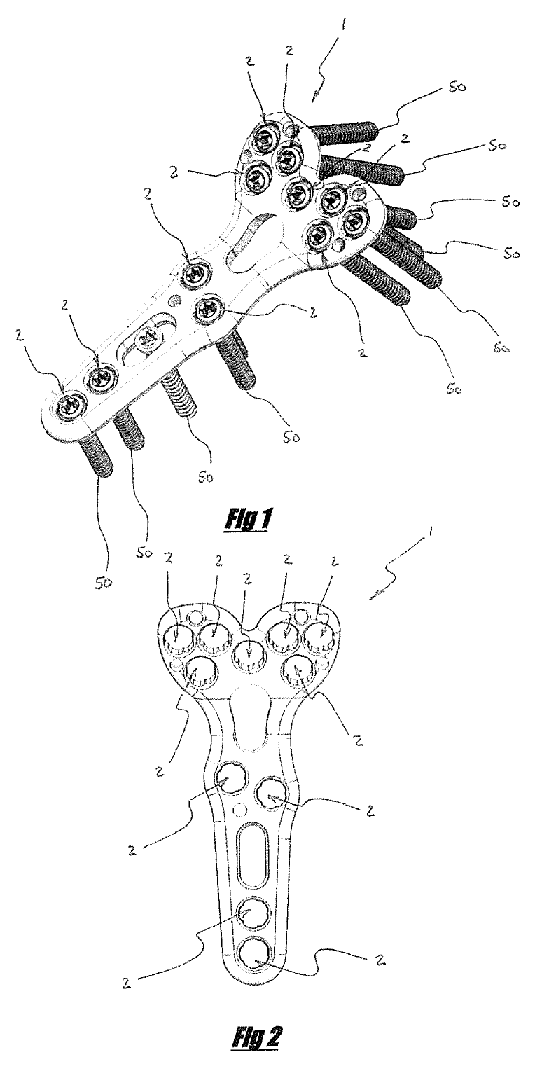

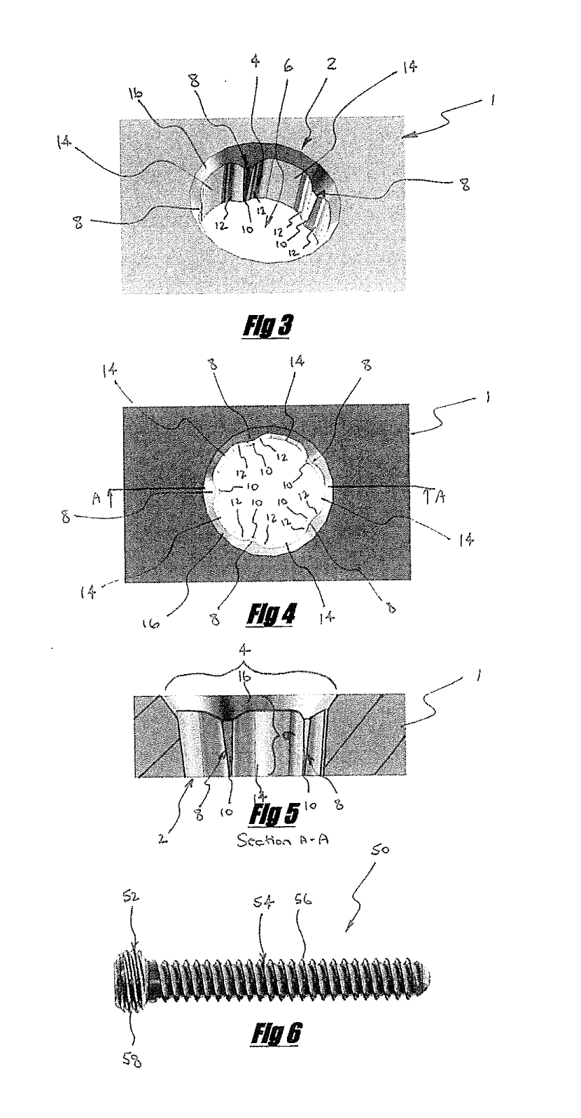

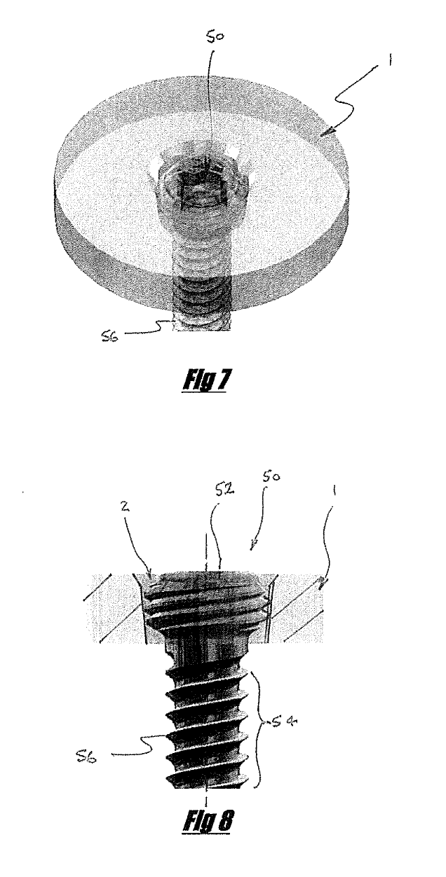

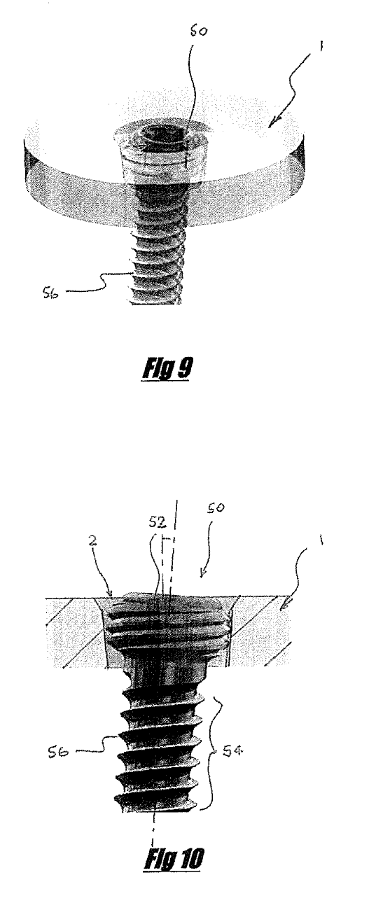

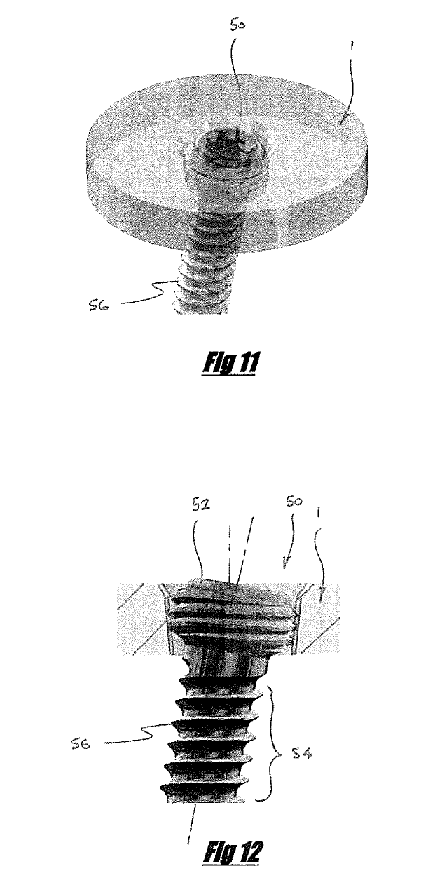

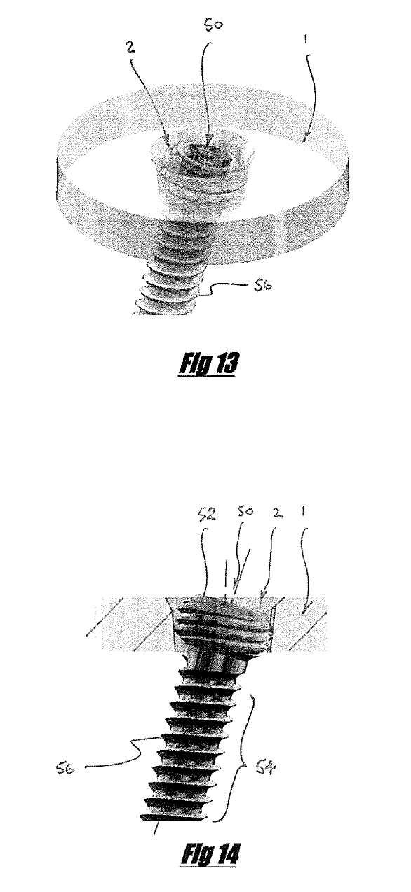

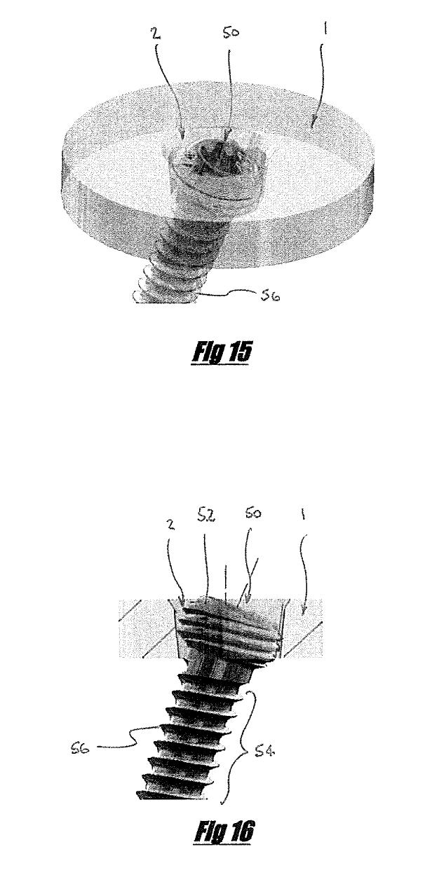

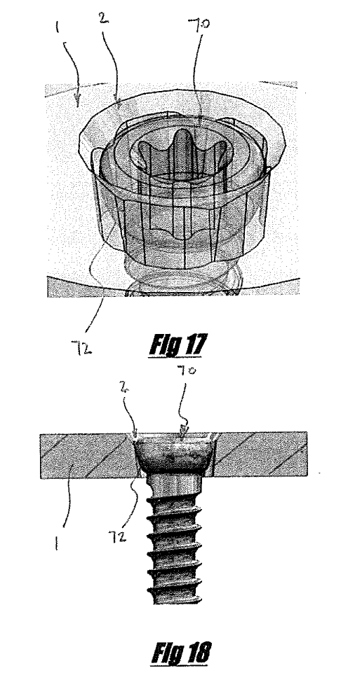

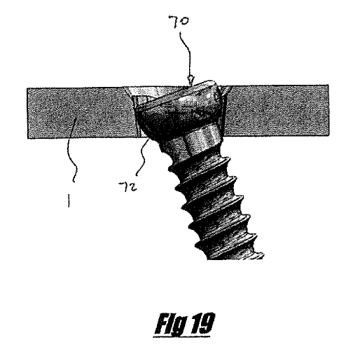

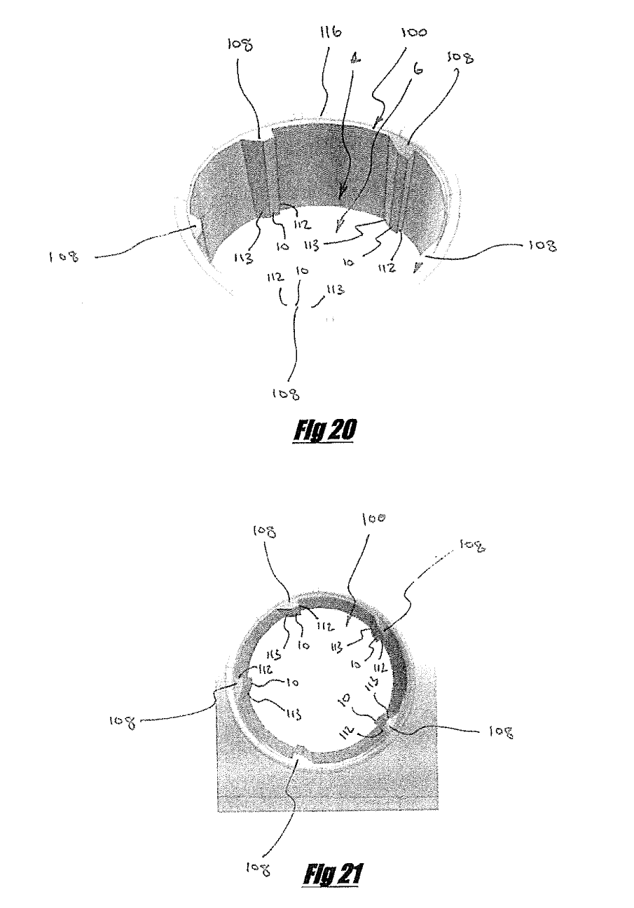

Referring now to FIGS. 1 through 5, there is shown a bone fixation system comprising a plurality of poly-axial (i.e. variable angle) screws 50 driven through through openings 2 in a bone fixation plate 1 and into a bone (not illustrated). The bone fixation plate 1 illustrated is one of the type used to repair distal radius fractures, however, it will be apparent to a person skilled in the art that the present invention is not limited to a bone fixation plate of this particular shape or type, but applicable to bone fixation plates of other shapes as well. A removable screw guide (not shown) may be secured to the bone fixation plate 1 by way of snap fit. In use, once all of the screws 50 have been inserted, this screw guide can be removed.

Bone fixation plates of this type are typically made from a titanium or a titanium alloy (such as Ti-6AI-4V) and are offered in a variety of sizes, where the number of through openings 2 depends on the size and purpose of the bone fixation plate.

Bone fixation plate 1 can be used on either of left or right hand sides of a body. The two holes in between the slots can be used on either wrist. The head of the plate is symmetrical and this is the section that adapts to the left or right hand depending on which drill guide is used; and hence the angulation of the screws is changed to fit the side being treated.

Referring now to FIGS. 3 through 5, where one of the through openings 2 in the bone fixation plate 1 is illustrated in detail.

The through opening 2 comprises a screw inlet 4, and a through bore 6 which tapers inwardly as it extends through the plate 1 from the screw inlet 4. The plate 1 further comprises five equi-spaced splines 8 which further define the through opening 2 by projecting into the through bore 6, each spline 8 extending longitudinally through the through opening 2, and comprises a top land 10 having a width which narrows as the spline 8 extends through the plate 1 from the screw inlet 4. Each spline 8 comprises a pair of sides 12, one side 12 either side of the top land 10, both of which blend into the through bore 6.

Each spline side 12 is a fillet which blends into a bottom land 14 which separates adjacent splines 8. Each spline side fillet 12 is concave and shaped like an arc of a circle, and each bottom land 14 is similarly concave and shaped like an arc of a circle.

A radius of each fillet arc will hereinafter be referred to as the ‘fillet arc radius’, and a radius of each bottom land arc will hereinafter be referred to as the ‘bottom land arc radius’. For each bottom land 14 and fillet 12, the bottom land arc radius is greater than a fillet arc radius.

The top land 10 of each spline 8 tapers inwardly as it extends through the plate 1 from the screw inlet 4. That is to say, the top land 10 of each spline 8 extends parallel to the through bore 6.

An edge of the plate 1 surrounding the screw inlet 4 comprises a lead-in portion 16 which tapers (although it could be rounded/radiused) both inwardly as it extends from the screw inlet 4 to the splines 8, and with a higher degree of taper (approximately 45 degrees) than the through bore (approximately 13 degrees). For very thin plates the lead-in 16 can be omitted, so as to maximise hole depth, without compromising angulation capability.

The through opening 2 and splines 8 may be formed using a computer controlled milling centre (i.e. a milling machine with automatic tool changers, tool magazines or carousels, CNC control, and coolant systems), which is firstly programmed to machine (bore) a tapered bore into the plate 1. Identical, equi-spaced portions of a side of this tapered bore are then relieved with a rotary cutting tool, leaving the five splines 8 as remnants of the original tapered bore. Then, the tapered lead-in portion 16 will be cut.

Forming the opening 2 and splines 8 in this way provides increased control of the spline 8 shape, and means that the splines are not “pointed”, as is the case if they are formed by the intersection of two angled holes.

Referring now to FIG. 6, where there is illustrated a variable angle non-locking screw 50 of the type disclosed in PCT/AU2013/000536.

The screw 50 comprises a head 52 and a shank 54. The head has a slot or socket via which it is driven by a tool. The screw 50 further includes an external bone engaging thread 56 located along the shank 54, to engage the screw 50 with a bone and thereby fix the bone fixation plate 1 with respect to the bone.

The head 52 of the screw 50 is securable to the bone fixation plate 1 by way of provision of one or more (three in this case) external locking threads 58.

The screw 50 material should be harder than the plate 1 material. By way of non-limiting examples, the following combinations of plate and screw materials may be used:

| Plate Material | Screw Material |
|---|---|
| Ti-grade 1, 2, 3, or 4 | Ti6AlV4 |
| 316LVM stainless steel | High Ni SS (ASTM 5832-9) |
| Ti6AlV4 | CoCr |
| Ti Grade 2 to ISO 5832-2 | Ti Grade 5 ISO 5832-3 |
| Titanium (any grade) | CoCr (eg ASTM F799) |
| Stainless steel 316L | SS ISO 5832-9 |

FIGS. 7 through 16 depict the bone fixation plate 1 and screw 50 in use, in combination. Initially, the screw 50 is inserted into the screw inlet 4 and through bore 6 and the bone engagement thread is screwed into the bone. Once the screw 50 is sufficiently inserted, the external locking thread (or threads) on the head 52 begins to come into engagement with the splines 8 of the through opening 2, and cut a portion of a thread or threads into the top land 10 of each of the five splines 8 and thereby secure the head of the screw 50 to the bone fixation plate 1. Further to this description, it should now be apparent that the top lands 10 provide a meaningful amount of area of the plate 1 material for the external locking thread on the head 52 of the screw 50 to find purchase in.

Because both the bore 6 and the splines 8 are tapered, the further the screw 50 descends into the plate 1 the deeper are the threads cut into the splines 8. When the screw driver cannot generate enough torque to drive the screw 50 through the plate 1 the screw is considered locked. The extent of the resulting plastic deformation of the splines 8 has the effect that unintended loosening of the screw 50 is not possible, as loosening is possible only with application of considerable force.

It will be apparent from the description provided herein, how the taper of through opening 2 and splines 8 provide for insertion of the screw 50 with varying degrees of inclination relative to a central axis of the through opening 2 in the plate 1. What is more, the tapered lead-in portion 16, and relieved areas between the splines 8 provide additional clearance for permitting inclination of the screw 50.

FIGS. 7 and 8 illustrate the screw 50 passing through a portion of the bone fixation plate 1 with no deviation (i.e. so that a longitudinal axis of the screw 50 is aligned with a central axis of the through opening 2).

FIGS. 9 and 10 illustrate the screw 50 passing through a portion of the bone fixation plate 1 with 5 degrees of deviation.

FIGS. 11 and 12 illustrate the screw 50 passing through a portion of the bone fixation plate 1 with 10 degrees of deviation.

FIGS. 13 and 14 illustrate the screw 50 passing through a portion of the bone fixation plate 1 with 15 degrees of deviation.

FIGS. 15 and 16 illustrate the screw 50 passing through a portion of the bone fixation plate 1 with 20 degrees of deviation.

FIGS. 17 through 19 illustrate a poly-axial non-locking screw 70 passing through a portion of the bone fixation plate 1. This poly-axial non-locking screw 70 comprises a head 72 with a generally bulbous underside which in use will bear against and cause plastic deformation of the splines 8, thereby becoming jammed between the splines 8.

While a through opening 2 comprising five equi-spaced splines 8 is illustrated in the Figures and described herein, it should be understood that the through opening 2 may comprise a differing number of splines 8 of differing geometry.

Referring now to FIGS. 20 and 21, where there is illustrated a through opening 100 in a bone fixation plate which has been manufactured by a 3D printing process, particularly one which allows 3D printing of metal alloys (such as Titanium). An advantage of 3D printing a bone fixation plate is that it is possible to produce through holes having geometries which cannot be produced using conventional machining techniques.

Those parts of the through opening 100 which are identical to corresponding parts shown in the through opening 2 of FIGS. 1 through 5, will be denoted by the same reference numerals and will not be described again in detail.

The through opening 100 comprises a screw inlet 4, and a through bore 6 which tapers inwardly as it extends through the plate 1 from the screw inlet 4. The plate 1 further comprises five equi-spaced splines 108 which further define the through opening 100 by projecting into the through bore 6, each spline 108 extending longitudinally through the through opening 100, and comprising a top land 10, and a pair of straight sides 112 and 113, one side 112 or 113 either side of the top land 10. The splines 108 of FIGS. 20 and 21 differ from those of FIGS. 1 through 5, in as much as they are asymmetric, with one spline side 112 being shorter than the other spline side 113, and more steeply inclined. The absence of fillets at the spline sides 112 and 113 results in the screw 70 cutting right through the splines 108, with its further rotation being stopped once it embeds itself in (or at least wedges itself against) the bottom lands 14 (i.e. against a wall of the through bore 6). In contrast, in the through opening 2 of FIGS. 1 through 5, the deeper the screw 70 cuts into the splines 8, the greater is the contact area between these, potentially halting the screw 70 before it comes into contact with the bottom lands 14. It is conceivable therefore that through opening 100 comprising splines 108, will increase locking and back out torque.

An edge of the plate 1 surrounding the screw inlet 4 comprises a lead-in portion 116 which is radiused inwardly as it extends from the screw inlet 4 to the splines 108.

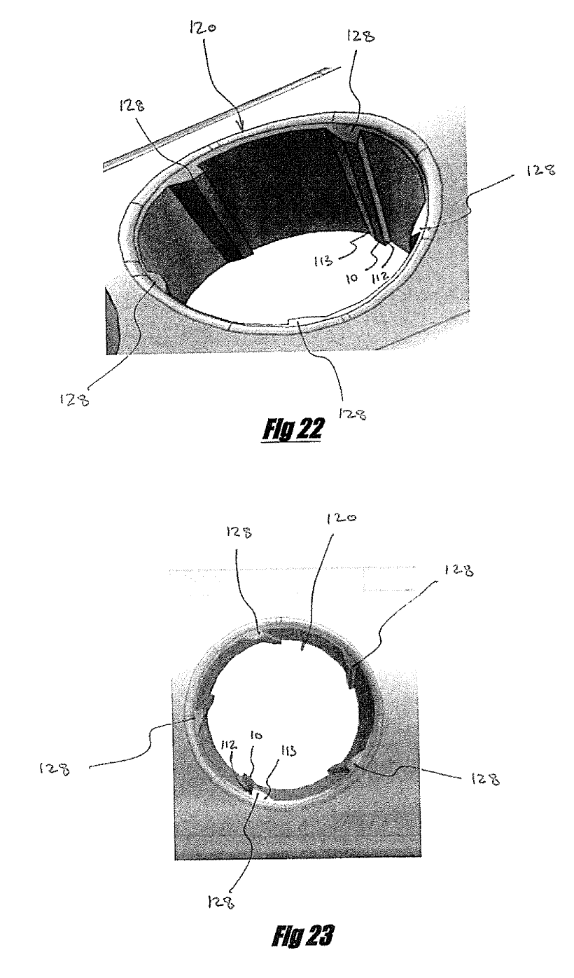

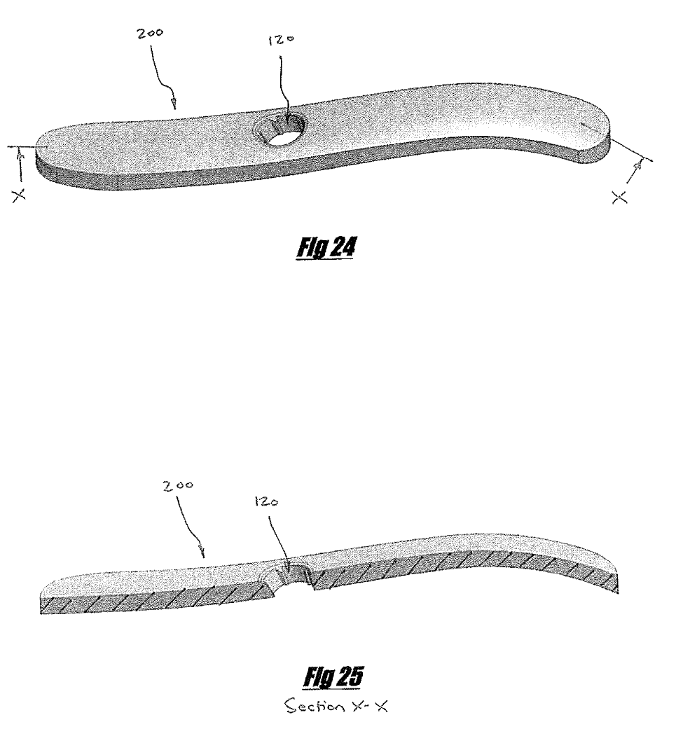

Referring now to FIGS. 22 and 23, where there is illustrated a further embodiment of a through opening in a bone fixation plate which had been manufactured by a 3D printing process.

Those parts of the through opening 120 which are identical to corresponding parts shown in the through opening 100 of FIGS. 20 and 21, will be denoted by the same reference numerals and will not be described again in detail.

The splines 128 of through opening 120 comprise a substantially similar cross-sectional shape to the splines 108 of through opening 100 in FIGS. 20 and 21, but differ inasmuch as the splines 128 do not longitudinally extend through by the most direct (and shortest) route, but extend (spiral) around the wall of the through bore 6 as they extend through the through opening 120.

In use, the screw 50 will wedge against the bottom lands 14, and splines 108 will improve locking of the screw 50 to the plate 1 by providing increased stability and resistance to lateral screw forces. The screw 50 will deform as it forces its way into the plate 1 and form sections of a conical shape which will, in effect, form a taper lock.

Referring now to FIGS. 24 and 25, where there is illustrated a further bone fixation plate 200 which had been manufactured by a 3D printing process, and which comprises a through opening 120. An additional advantage of 3D printing a bone fixation plate is that it is possible to produce plates comprising complex shapes such as curves, and combinations of curves.

Any of the hole geometries described above can be applied to curved and/or bent sections of plate 200. Additionally the axis of the through hole does not need to be perpendicular to the plate 200 surface.

This is very applicable to some fixation sites that will require a variable axis screw 50 to be inserted at angles greater than 20 degrees, yet still maintain a variety of angulation possibilities in relation to the hole axis.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. A bone fixation system comprising:
a screw having a head; and
a plate comprising:
a through opening, the through opening comprising a screw inlet, and a through bore which tapers inwardly as it extends through the plate from the screw inlet and
a plurality of splines which further define the through opening by projecting into the through bore, each spline extending longitudinally through the through opening and comprising a top land which extends from the screw inlet along the spline,
wherein, in use, at least a portion of the head of the screw will engage with the top lands of the splines.

2. The bone fixation system of claim 1, wherein each top land comprises a width which narrows as the spline extends through the plate from the screw inlet.

3. The bone fixation system of claim 1, wherein the top land of each spline tapers inwardly as it extends through the plate from the screw inlet.

4. The bone fixation system of claim 1, wherein each spline deepens as it extends longitudinally through the through opening.

5. The bone fixation system of claim 1, wherein an edge of the plate surrounding the screw inlet comprises a lead-in portion which tapers both inwardly as it extends from the screw inlet to the splines, and with a higher degree of taper than the through bore.

6. The bone fixation system of claim 1, wherein each spline comprises a pair of sides on either side of the top land, both sides of each spline configured to blend into the through bore.

7. The bone fixation system of claim 1, wherein the screw is comprised of a harder material than the plate.

8. The bone fixation system of claim 1, wherein the screw is a poly-axial locking screw

9. The bone fixation system of claim 1, wherein the screw is a poly-axial non-locking screw.

10. A plate for a bone fixation system comprising:
a through opening comprising:
a screw inlet; and
a through bore which tapers inwardly as it extends through the plate from the screw inlet; and
a plurality of equi-spaced splines which further define the through opening by projecting into the through bore, each spline extending longitudinally through the through opening and comprising a top land which extends from the screw inlet along the spline.

11. The plate of claim 10, wherein each top land comprises a width which narrows as the spline extends through the plate from the screw inlet.

12. The plate of claim 10, wherein the top land of each spline tapers inwardly as it extends through the plate from the screw inlet.

13. The plate of claim 10, wherein the top land of each spline extends substantially parallel to the through bore.

14. The plate of claim 10, wherein an edge of the plate surrounding the screw inlet comprises a lead-in portion which tapers both inwardly as it extends from the screw inlet to the splines, and with a higher degree of taper than the through bore.

15. The plate of claim 10, wherein each spline comprises a pair of sides on either side of the top land, where each of the pair of sides is a fillet which blends into a bottom land which separates adjacent splines.

16. The plate of claim 15, wherein the fillet is concave and arcuate, and the bottom land is concave and arcuate.

17. The plate of claim 16, wherein a bottom land arc radius is greater than a fillet arc radius.

18. The plate of claim 10, wherein the plate is manufactured by a 3D printing process.

19. A method for forming the plate of claim 10, the method comprising:
creating the through bore in the plate; and then
relieving the wall of the through bore identically at a plurality of equi-spaced locations so as to form the splines.

20. A bone fixation system comprising:
a screw having a head; and
a plate, wherein the screw is comprised of a harder material than the plate, and wherein the plate comprises:
a through opening, the through opening comprising a screw inlet, and a through bore which tapers inwardly as it extends through the plate from the screw inlet; and
five equi-spaced splines which further define the through opening by projecting into the through bore, each spline extending longitudinally through the through opening and comprising a top land which extends from the screw inlet along the spline,
wherein, in use, at least a portion of the head of the screw comprises a triple start thread for cutting into the top lands of the splines.
